(19) United States
(12) Patent Application Publication
(10) Pub. No.: US 2025/0019500 A1
(43) Pub. Date: Jan. 16, 2025

(54) CROSSLINKED HYDROGELS WITH ENHANCED RADIOPACITY FOR MEDICAL APPLICATIONS

(71) Applicant: Boston Scientific Scimed, Inc., Maple Grove, MN (US)

(72) Inventors: Rick Dorn, Plymouth, MN (US); Joseph Thomas Delaney, JR., Minneapolis, MN (US)

(73) Assignee: Boston Scientific Scimed, Inc., Maple Grove, MN (US)

(21) Appl. No.: 18/763,649

(22) Filed: Jul. 3, 2024

Related U.S. Application Data
(60) Provisional application No. 63/512,263, filed on Jul. 6, 2023.

(51) Int. Cl. C08J 3/075; A61L 27/18; A61L 27/52; A61L 27/58; C08K 3/08

(52) U.S. Cl. CPC C08J 3/075; A61L 27/18; A61L 27/52; A61L 27/58; C08K 3/08; A61L 2400/06; C08J 2300/202; C08K 2003/0831; C08K 2201/011

(57) ABSTRACT

The present disclosure pertains to systems for forming hydrogel compositions that comprise a reactive polymer comprising a plurality of hydrophilic polymer segments and a plurality of first reactive moieties and gold nanoparticles, wherein the system is configured to deliver the reactive polymer and the gold nanoparticles under conditions such that covalent crosslinks are formed between the reactive polymer and the gold nanoparticles. The present disclosure also pertains to methods of treatment that comprise administering to a subject a mixture that comprises a reactive polymer comprising a plurality of hydrophilic polymer segments and a plurality of first reactive moieties and gold nanoparticles, under conditions such that the reactive polymer and the gold nanoparticles crosslink after administration, and to radiopaque crosslinked hydrogel compositions that comprise a crosslinked reaction product of a reactive polymer comprising a plurality of hydrophilic polymer segments and a plurality of first reactive moieties and gold nanoparticles.

### CROSS REFERENCE TO RELATED APPLICATIONS

The application claims the benefit of U.S. Provisional Patent Application Ser. No. 63/512,263 filed on Jul. 6, 2023, the disclosure of which is incorporated herein by reference.

### FIELD

The present disclosure relates radiopaque hydrogels and to crosslinkable systems for forming radiopaque hydrogels, among other aspects. The radiopaque hydrogels and crosslinkable systems for forming the same are useful, for example, in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

Alternative strategies for forming iodine-labelled crosslinked hydrogels that provide enhanced radiopacity while maintaining or improving crosslink density per polymer molecule are desired.

### SUMMARY

In some aspects, the present disclosure pertains to systems for forming hydrogel compositions that comprise (a) a reactive polymer comprising a plurality of hydrophilic polymer segments and a plurality of first reactive moieties and (b) gold nanoparticles, wherein the system is configured to deliver the reactive polymer and the gold nanoparticles under conditions such that covalent crosslinks are formed between the reactive polymer and the gold nanoparticles.

In some embodiments, the reactive polymer is a multi-arm polymer that comprises three or more polymer arms linked to a core region, each arm comprising one of the plurality of hydrophilic polymer segments and one of the plurality of first reactive moieties. In some of these embodiments, the reactive polymer is a multi-arm polymer that comprises three or more polymer arms linked to a core region, each arm comprising a cyclic anhydride residue disposed between one of the plurality of hydrophilic polymer segments and one of the plurality of first reactive moieties. The core region may comprise, for example, polyol residue, among other possibilities.

In some embodiments, which can be used in conjunction with the above aspects and embodiments, the hydrophilic polymer segments are selected from polyalkylene oxide segments, polyester segments, polyoxazoline segments, polydioxanone segments, and polypeptide segments.

In some embodiments, which can be used in conjunction with the above aspects and embodiments, each of the hydrophilic polymer segments contains between 10 and 1000 monomer residues.

In some embodiments, which can be used in conjunction with the above aspects and embodiments, the gold nanoparticles range from 1 nm to 2 micrometers in longest dimension.

In some embodiments, which can be used in conjunction with the above aspects and embodiments, the first reactive moieties comprise thiol groups and the gold nanoparticles comprise a stabilization agent that exhibits a lower affinity for gold than the thiol groups. In some of these embodiments, the stabilization agent is a surfactant.

In some embodiments, which can be used in conjunction with the above aspects and embodiments, the gold nanoparticles comprise a plurality of second reactive moieties that form covalent crosslinks with the first reactive moieties. For example, (a) the first reactive moieties may comprise a cyclic imide ester group and the second reactive moieties may comprise a primary amine, thiol or hydroxyl group, (b) the first reactive moieties may comprise a primary amine, thiol or hydroxyl group and the second reactive moieties may comprise a cyclic imide ester group, (c) the first reactive moieties may comprise a strained alkyne group and the second reactive moieties may comprise an azide group, (d) the first reactive moieties may comprise an azide group and the second reactive moieties may comprise a strained alkyne group, (e) the first reactive moieties may comprise a strained alkene group and the second reactive moieties may comprise tetrazine a group, or (f) the first reactive moieties may comprise a tetrazine group and the second reactive moieties may comprise a strained alkene group.

In some embodiments, which can be used in conjunction with the above aspects and embodiments, the system further comprises a delivery device.

In some aspects, the present disclosure provides methods of treatment that comprise administering to a subject a mixture that comprises a reactive polymer and gold nanoparticles in accordance with any of the above aspects and embodiments, under conditions such that the reactive polymer and the gold nanoparticles crosslink after administration.

In some embodiments, the methods comprise administering to the subject a first fluid composition that comprises the reactive polymer and a second fluid composition that comprises the gold nanoparticles.

In some embodiments, the methods comprise administering to the subject a first fluid composition that comprises the reactive polymer and the gold nanoparticles and a second fluid composition that comprises an accelerant that accelerates formation of the covalent crosslinks.

In some embodiments, the first fluid composition and the second fluid composition are delivered using a double barrel syringe.

In some aspects, the present disclosure provides radiopaque crosslinked hydrogel compositions that comprise a crosslinked reaction product of reactive polymer and gold nanoparticles in accordance with any of the above aspects and embodiments.

In some aspects, the present disclosure provides methods of treatment that comprise administering the radiopaque crosslinked hydrogel compositions to patients.

Potential benefits associated with the present disclosure include one or more of the following: radiocontrast is maintained, crosslink density is enhanced, and in vivo persistence is obtained.

The above and other aspects, embodiments, features and benefits of the present disclosure will be readily apparent from the following detailed description.

### BRIEF DESCRIPTION OF THE DRAWINGS

FIG. 1 schematically illustrates a reactive multi-arm polymer, in accordance with an embodiment of the present disclosure.

FIG. 2 schematically illustrates a method of forming a reactive gold nanoparticle, in accordance with an embodiment of the present disclosure.

FIGS. 3A-3D schematically illustrate methods of forming reactive polymers, in accordance with four embodiments of the present disclosure.

FIGS. 4A-4C schematically illustrate methods of forming covalent linkages, in accordance with three embodiments of the present disclosure.

FIG. 5 schematically illustrates a method of forming a crosslinked product by covalently linking a reactive multi-arm polymer and reactive gold nanoparticles, in accordance with an embodiment of the present disclosure.

FIG. 6 schematically illustrates a method of forming a reactive thiol-containing polymer, in accordance an embodiment of the present disclosure.

FIG. 7 schematically illustrates a method of forming a crosslinked product by covalently linking a reactive thiol-containing multi-arm polymer and stabilized gold nanoparticles, in accordance with an embodiment of the present disclosure.

FIG. 8 illustrates a delivery device, in accordance with an embodiment of the present disclosure.

FIG. 9 illustrates a delivery device, in accordance with another embodiment of the present disclosure.

### DETAILED DESCRIPTION

In some aspects, the present disclosure provides a system that comprises (a) a reactive polymer comprising first reactive moieties and (b) gold nanoparticles, wherein the system is configured to deliver the reactive polymer and the gold nanoparticles under conditions such that covalent crosslinks are formed between the reactive polymer and the gold nanoparticles.

In some embodiments, the system comprises (a) a reactive polymer comprising first reactive moieties and (b) reactive gold nanoparticles comprising second reactive moieties, wherein the first reactive moieties and the second reactive moieties covalently crosslink with one another to form a crosslinked reaction product.

In some embodiments, the system comprises (a) a reactive polymer comprising first reactive moieties that comprise thiol groups and (b) gold nanoparticles that are stabilized with a stabilization agent that exhibits a lower affinity for gold than the thiol groups, wherein the thiol groups crosslink with the gold nanoparticles to form a crosslinked reaction product.

Particular examples of first and second reactive moieties include the following among others (a) first reactive moieties that comprise electrophilic groups and second reactive moieties that comprise nucleophilic groups, or vice versa, (b) first reactive moieties that comprise strained alkyne groups and second reactive moieties that comprise azide groups, or vice versa, (c) first reactive moieties that comprise strained alkene groups and second reactive moieties that comprise tetrazine groups, or vice versa.

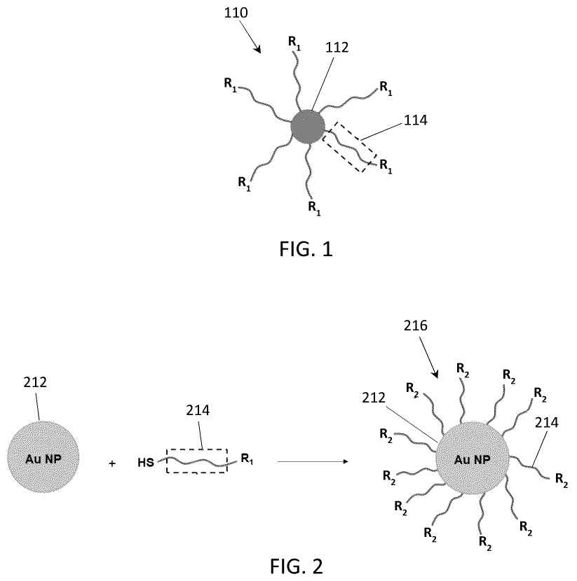

Referring now to FIG. 1, reactive polymers in accordance with the present disclosure include reactive multi-arm polymers 110 that comprise a plurality of polymer arms linked to a core region 112, at least a portion of the arms comprising a hydrophilic polymer segment 114. One end of the hydrophilic polymer segment 114 is covalently linked to the core region 112 and an opposite end of the hydrophilic polymer segment 114 is covalently linked to a first reactive moiety R₁.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a first reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

First reactive moieties R₁ include moieties that comprise electrophilic groups, moieties that comprise nucleophilic groups, moieties that comprise strained alkyne groups, moieties that comprise strained alkene groups, moieties that comprise azide groups, moieties that comprise tetrazine groups, and moieties that comprise thiol groups.

Electrophilic groups may be selected, for example, from cyclic imide ester groups, such as succinimide ester groups,

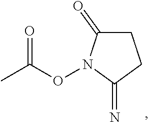

maleimide ester groups, glutarimide ester groups, diglycolimide ester groups, phthalimide ester groups, and bicyclo[2.2.1]hept-5-ene-2,3-dicarboxylic acid imide ester groups,

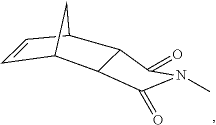

imidazole ester groups, imidazole carboxylate groups and benzotriazole ester groups, among other possibilities. Nucleophilic groups may be selected, for example, from primary amine groups, thiol groups and hydroxyl groups, among other possibilities. Strained alkyne groups may be selected, for example, from (1R,8S,9s)-bicyclo[6.1.0]non-4-yn-9-yl groups,

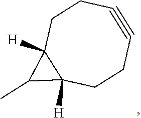

and dibenzocyclooctyne groups, among other possibilities. Strained alkene groups may be selected, for example, from cyclooct-4-en-1-yl groups,

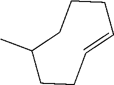

among other possibilities.

The electrophilic groups, nucleophilic groups, strained alkyne groups, strained alkene groups, azide groups, tetrazine groups, or thiol groups may be linked to the hydrophilic polymer segment 114 through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ω-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ω-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 10 monomer units or less to 1000 monomer units or more, for example, ranging from 5 to 10 to 20 to 50 to 100 to 200 to 500 to 1000 to 2000 monomer units or more (in other words, having a number of monomer units ranging between any two of the preceding values).

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The Ts cage-like silicon-oxygen cores are widely studied and have the formula [RSiO_{3/2}]₈, or equivalently R₈Si₈O₁₂. Such a structure is shown here:

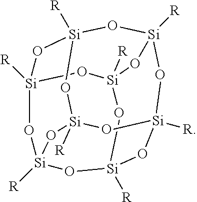

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

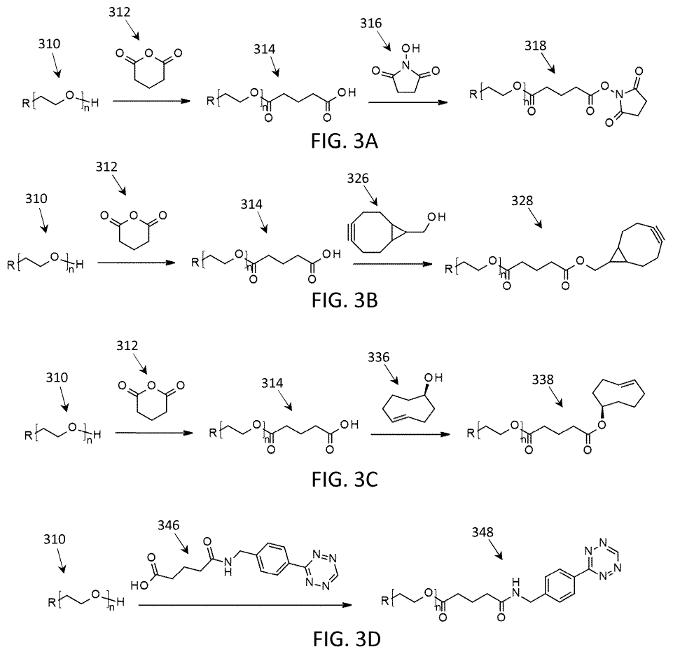

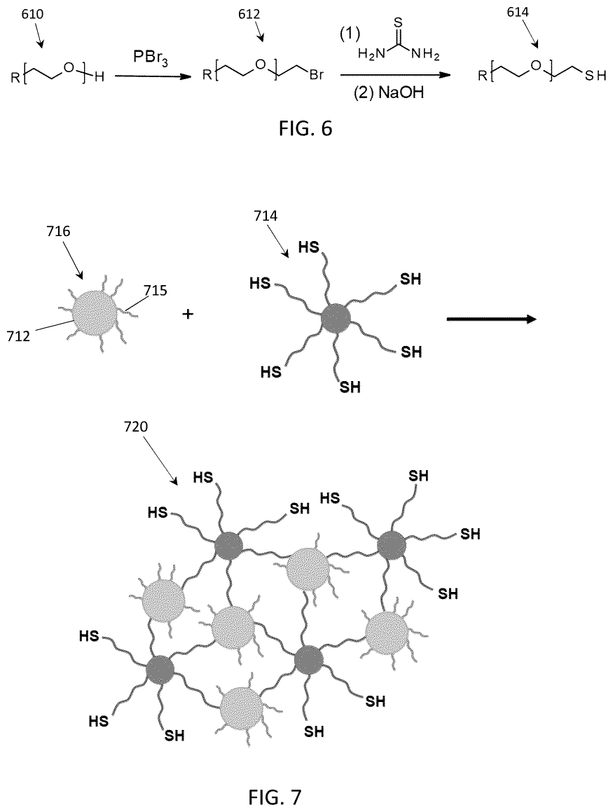

With reference now to FIGS. 3A-3C, a cyclic anhydride, specifically glutaric anhydride 312, is reacted with a hydroxy-terminated precursor multi-arm hydrophilic polymer, specifically a hydroxy-terminated precursor multi-arm polyethylene oxide (PEO) 310, where R corresponds to a core, to form an acid end-capped multi-arm polymer, specifically glutaric-acid-end-capped multi-arm polyethylene oxide (PEO) 314. (It is noted that although only one arm of the multi-arm polyethylene oxide 310 is shown attached to the core R in FIGS. 3A-3C, as well as in FIG. 3D and FIG. 6 described below, it is to be understood that additional polymer arms are present.) In FIGS. 3A-3D and FIG. 6, n is an integer and may have a value ranging from 1 to 1000 or more.

A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, malcimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In a particular embodiment shown in FIG. 3A, glutaric acid end-capped multi-arm PEO 314 is then reacted with N-hydroxysuccinimide 316 in the presence of a coupling agent to form a succinimidyl-glutarate-end-capped multi-arm PEO 318.

In some embodiments, a strained alkyne group may be linked to the carboxylic-acid-terminated precursor polymer. In a particular embodiment shown in FIG. 3B, glutaric acid end-capped multi-arm PEO 314 is reacted with a hydroxyl-substituted strained alkyne such as (1R,8S,9s)-bicyclo[6.1.0]non-4-yn-9-ylmethanol 326 in an ester coupling reaction in the presence of a suitable coupling agent to produce (1R,8S,9s)-bicyclo[6.1.0]non-4-yn-9-yl-glutarate-end-capped multi-arm PEO 328. In an alternative embodiment (not shown), acid end-capped multi-arm PEO is reacted with an amine-substituted strained alkyne such as (1R,8S,9s)-bicyclo[6.1.0]non-4-yn-9-ylmethamine in an amide coupling reaction in the presence of a suitable coupling agent to produce a multi-arm PEO in which (1R,8S,9s)-bicyclo[6.1.0]non-4-yn-9-yl groups are coupled to the polymer arms through an amide group and a hydrolysable ester group.

In some embodiments, a strained alkene group may be linked to the carboxylic-acid-terminated precursor polymer. In a particular embodiment shown in FIG. 3C, glutaric acid end-capped multi-arm PEO 314 is reacted with a hydroxyl-substituted strained alkene such as cyclooct-4-en-1-ol 336 in an ester coupling reaction in the presence of a suitable coupling agent to produce cyclooct-4-en-1-yl-glutarate-end-capped multi-arm PEO 338. In an alternative embodiment (not shown), acid end-capped multi-arm PEO is reacted with an amine-substituted strained alkene such as cyclooct-4-en-1-amine in an amide coupling reaction in the presence of a suitable coupling agent to produce a multi-arm PEO in which cyclooct-4-en-1-yl groups are coupled to the polymer arms through an amide group and a hydrolysable ester group.

In some embodiments, a tetrazine group may be linked to a hydroxyl-terminated precursor polymer. For example, a tetrazine based acid may be coupled to a hydroxyl-terminated precursor polymer in an ester coupling reaction in the presence of a suitable coupling agent. In a particular embodiment shown in FIG. 3D, a tetrazine-terminated polymer is prepared by coupling a hydroxy-terminated precursor multi-arm polyethylene oxide (PEO) 310 with 5-[4-(1,2,4,5-tetrazin-3-yl)benzylamino]-5-oxopentanoic acid in the presence of a carbodiimide coupling agent to produce 5-[4-(1,2,4,5-tetrazin-3-yl)benzylamino]-5-oxopentanoate-end-capped multi-arm PEO 338. It is noted that the reactive tetrazine group is linked to the multi-arm polymer through a hydrolysable ester group.

In some embodiments, a thiol group may be linked to a hydroxyl-terminated precursor polymer. For example, and with reference to FIG. 6, a hydroxyl terminated multi-arm polymer 610 can be converted to an alkyl bromide terminated multi-arm polymer 612 via a reaction with phosphorus tribromide (PBr₃). The alkyl bromide terminated multi-arm polymer 612 may then be reacted with thiourea (SC(NH₂)₂), followed by hydrolysis with an aqueous base, to form a thiol end-capped multi-arm polymer 614. As detailed below, such thiol end-capped polymers can form a hydrogel directly with gold nanoparticles in-vivo due to the high affinity of thiols for gold.

The strategies shown in FIGS. 3A-3D and FIG. 6 are widely applicable to hydroxy-terminated polymers having hydrophilic polymer segments besides PEO segments, such as those disclosed above.

As previously noted, in some embodiments, the present disclosure provides systems that comprise (a) a reactive polymer comprising first reactive moieties as described above and (b) reactive gold nanoparticles comprising second reactive moieties. Such reactive gold nanoparticles will now be described.

Referring now to FIG. 2, a reactive gold nanoparticle 216 in accordance with the present disclosure is shown that comprises a plurality of polymer chains linked to a gold nanoparticle 212, the polymer chains comprising a hydrophilic polymer segment 214 and a second reactive moiety R₂. One end of the hydrophilic polymer segment 214 is covalently linked to the gold nanoparticle 212 and an opposite end of the hydrophilic polymer segment 214 is covalently linked to the second reactive moiety R₂.

Second reactive moieties R₂ for use in the present disclosure include moieties that comprise electrophilic groups, moieties that comprise nucleophilic groups, moieties that comprise strained alkyne groups, moieties that comprise strained alkene groups, moieties that comprise azide groups, and moieties that comprise tetrazine groups. Second reactive moieties R₂ may be linked to the hydrophilic polymer segment 214 through any suitable linking moiety, which may be selected, for example, from an alkyl group, ether group, ester group, amide group, amine group, carbonate group, or combinations of two or more of the foregoing groups, among others. In certain embodiments, the linking moiety comprises a hydrolysable ester group.

The gold nanoparticles may be solid gold particles, or the gold nanoparticles may comprise a gold shell surrounding a core particle that is formed from a metal other than gold, for example, tantalum or tungsten, among others. The gold in the nanoparticles may be substantially pure gold or may by a gold alloy (e.g., gold alloyed with one or more metals selected from silver, platinum, copper, titanium, rhodium, palladium, zinc, nickel, iron, and aluminum).

The gold nanoparticles may be provided in a variety of shapes, including spherical, rod-shaped, plate-shaped and irregularly shaped. The size of the gold nanoparticle may be chosen for the specific imaging application and desired radiopacity. See Xi, D. et al., Gold nanoparticles as computerized tomography (CT) contrast agents. RSC Adv. 2012, 2 (33), 12515-12524. For example, where the imaging is X-ray based, the particles may range from 1 nm to 2000 nm, for example, ranging anywhere from 1 nm to 2 nm to 5 nm to 10 nm to 20 nm to 50 nm to 100 nm to 200 nm to 500 nm to 1000 nm to 2000 nm, in longest dimension (e.g., diameter for a sphere, length for a rod, greatest width for a platelet, etc.). As another example, where the imaging is near-IR fluorescence spectrometry-based, the particles may range from 20 nm to 500 nm, for example, ranging anywhere from 20 nm to 50 nm to 100 nm to 200 nm to 500 nm in longest dimension.

Gold nanoparticles may be surface functionalized with a hydrophilic polymer that comprises a hydrophilic polymer segment 214 having a thiol group (—SH group) attached to one end and a second reactive moiety R₂ attached to the other end. Surface functionalization of the gold nanoparticles occurs readily when the thiol terminated polymer is added to a solution of gold nanoparticles. See, e.g., Gao, J. et al., Colloidal Stability of Gold Nanoparticles Modified with Thiol Compounds: Bioconjugation and Application in Cancer Cell Imaging. Langmuir 2012, 28 (9), 4464-4471; Zopes, D. et al., Improved Stability of “Naked” Gold Nanoparticles Enabled by in Situ Coating with Mono and Multivalent Thiol PEG Ligands. Langmuir 2013, 29 (36), 11217-11226; Hinterwirth, H. et al., Quantifying Thiol Ligand Density of Self-Assembled Monolayers on Gold Nanoparticles by Inductively Coupled Plasma-Mass Spectrometry. ACS Nano 2013, 7 (2), 1129-1136.

Hydrophilic polymer segments for use in forming reactive gold nanoparticles can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ω-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, among others, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ω-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, or polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the reactive gold nanoparticles of the present disclosure typically contain between 5 or less and 100 monomer units or more, for example, ranging from 2 to 5 to 10 to 20 to 50 to 100 to 200 monomer units in length.

As previously noted, in some aspects, the present disclosure provides a radiopaque hydrogel that comprises a crosslinked reaction product of (a) a reactive polymer as described hereinabove and (b) gold nanoparticles as described hereinabove. In various embodiments, the reactive polymer will have three or more first reactive end groups (e.g., three or more arms terminated with a first reactive moiety R₁) and the gold nanoparticles will have two or more second reactive end groups (e.g., two or more polymer chains terminated with a second reactive moiety R₂).

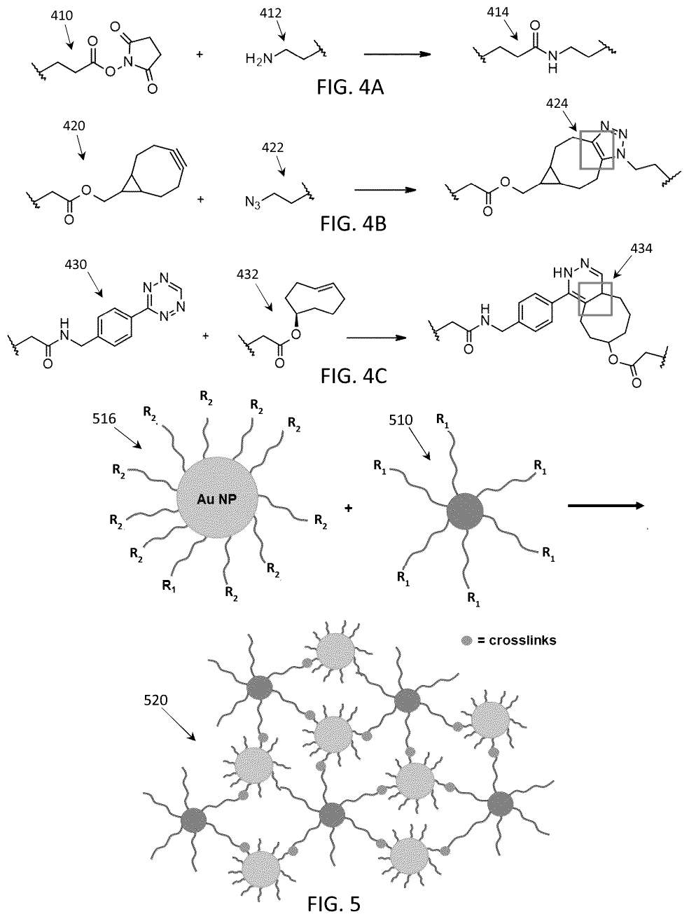

Three specific examples of covalently crosslinking reactions between first and second reactive moieties (e.g., between a reactive moiety R₁ and second reactive moiety R₂) are shown in FIGS. 4A-4C.

FIG. 4A shows a covalent crosslinking reaction between a cyclic amide ester group, specifically, a succinimide ester group 410 and a primary amine group 412, whereby an amide linking group 414 is formed. In this scheme, the first reactive moiety may comprise the cyclic amide ester group and the second reactive moiety may comprise the primary amine group, or the first reactive moiety may comprise the primary amine group and the second reactive moiety may comprise the cyclic amide ester group.

FIG. 4B shows the formation of a cyclooctatriazole covalent linkage 424 through a strain-promoted azide-alkyne cycloaddition click chemistry (SPACC) reaction between a strained alkyne group, specifically, a (1R,8S,9s)-bicyclo[6.1.0]non-4-yn-9-yl-ester group 420 with an azide group 422. The box in FIG. 4B denotes where the new covalent bonding is formed. In this scheme, the first reactive moiety may comprise the strained alkyne group and the second reactive moiety may comprise the azide group, or the first reactive moiety may comprise the azide group and the second reactive moiety may comprise the strained alkyne group.

FIG. 4C shows the formation of a cyclooctapyridazine covalent linkage 434 through a strain-promoted tartrazine ligation coupling reaction between a tetrazine group, specifically, a 1,2,4,5-tetrazin-3-yl group 430 and a strained alkene group, specifically, a cyclooct-4-en-1-yl-ester group 432. The box in FIG. 4C denotes where the new covalent bonding is formed. In this scheme, the first reactive moiety may comprise the strained alkene group and the second reactive moiety may comprise the tetrazine group, or the first reactive moiety may comprise the tetrazine group and the second reactive moiety may comprise the strained alkene group.

A crosslinking reaction is illustrated schematically in FIG. 5, which shows a reactive multi-arm polymer 510 having a plurality of first reactive moieties R₁ being covalently crosslinked with reactive gold nanoparticles 516 having a plurality of reactive moieties R₂ to form a crosslinked reaction product, specifically, a crosslinked radiopaque hydrogel product 520.

As noted above, in various aspects, the present disclosure provides a system that comprises (a) a reactive polymer comprising reactive thiol groups and (b) gold nanoparticles that are stabilized with one or more stabilization agents that exhibit a lower affinity for gold than thiol groups, wherein the thiol groups crosslink with the gold nanoparticles to form gold-sulfur bonds.

Examples of a reactive polymers that comprise reactive thiol groups are described above, including multi-arm polymers that comprise a plurality of polymer arms linked to a core region, the polymer arms comprising a hydrophilic polymer segment, where one end of the hydrophilic polymer segment is covalently linked to the core region and an opposite end of the hydrophilic polymer segment is covalently linked to a reactive moiety comprising a thiol group. A particular example is described above in conjunction with FIG. 6.

Gold nanoparticles are described above. Stabilization agents that can be used to stabilize gold nanoparticles include surfactants such as water-soluble carboxyl, amine sulfate, and phosphine ligands, among others, for example, sodium citrate and sodium dodecyl sulfate. See Rouhana, L. L., et al., Aggregation-Resistant Water-Soluble Gold Nanoparticles. Langmuir 2007, 23 (26), 12799-12801, Warner, M. G., et al., Small, Water-Soluble, Ligand-Stabilized Gold Nanoparticles Synthesized by Interfacial Ligand Exchange Reactions. Chem Mater 2000, 12 (11), 3316-3320; Schulz, F. et al., Little Adjustments Significantly Improve the Turkevich Synthesis of Gold Nanoparticles. Langmuir 2014, 30 (35), 10779-10784.

In various aspects, the present disclosure provides a radiopaque hydrogel that comprises a reaction product of (a) a reactive polymer having three or more thiol groups (e.g., having three or more polymer arms terminated with a thiol containing moiety) and (b) stabilized gold nanoparticles.

A crosslinking reaction is shown schematically in FIG. 7, in which a reactive multi-arm polymer 714, which has a plurality of polymer arms that are terminated with reactive thiol groups, is crosslinked with stabilized gold nanoparticles 716, for example, gold nanoparticles 712 that are stabilized with a stabilization agent 715, to form a crosslinked reaction product, specifically, a radiopaque hydrogel product 720.

In some embodiments, the present disclosure provides a crosslinked reaction product of a reactive polymer comprising first reactive moieties that comprise electrophilic groups and gold nanoparticles comprising second reactive moieties that comprise nucleophilic groups, or a crosslinked reaction product of a reactive polymer comprising first reactive moieties that comprise nucleophilic groups and gold nanoparticles comprising second reactive moieties that comprise electrophilic groups. In some embodiments, the present disclosure provides a system for forming a crosslinked reaction product by combining a reactive polymer comprising first reactive moieties that comprise electrophilic groups with gold nanoparticles comprising second reactive moieties that comprise nucleophilic groups, or by combining a reactive polymer comprising first reactive moieties that comprise nucleophilic groups and gold nanoparticles comprising second reactive moieties that comprise electrophilic groups. The reactive polymer and the gold nanoparticles are combined under conditions such that the electrophilic and nucleophilic groups crosslink with one another. In certain embodiments, those conditions comprise an environment having a basic pH, for example, a pH ranging from about 9 to about 11. Such crosslinked reaction products can be formed in vivo or ex vivo.

In some embodiments, the present disclosure provides a crosslinked reaction product of a reactive polymer comprising first reactive moieties that comprise strained alkyne groups and gold nanoparticles comprising second reactive moieties that comprise azide groups, or a crosslinked reaction product of a reactive polymer comprising first reactive moieties that comprise azide groups and gold nanoparticles comprising second reactive moieties that comprise strained alkyne groups. In some embodiments, the present disclosure provides a system for forming a crosslinked reaction product by combining a reactive polymer comprising first reactive moieties that comprise strained alkyne groups with gold nanoparticles comprising second reactive moieties that comprise azide groups, or by combining a reactive polymer comprising first reactive moieties that comprise azide groups and gold nanoparticles comprising second reactive moieties that comprise strained alkyne groups. The reactive polymer and the gold nanoparticles are combined under conditions such that the strained alkyne and azide groups crosslink with one another. Such crosslinked reaction products can be formed in vivo or ex vivo.

In some embodiments, the present disclosure provides a crosslinked reaction product of a reactive polymer comprising first reactive moieties that comprise strained alkyne groups and gold nanoparticles comprising second reactive moieties that comprise tetrazine groups, or a crosslinked reaction product of a reactive polymer comprising first reactive moieties that comprise tetrazine groups and gold nanoparticles comprising second reactive moieties that comprise strained alkyne groups. In some embodiments, the present disclosure provides a system for forming a crosslinked reaction product by combining a reactive polymer comprising first reactive moieties that comprise strained alkyne groups with gold nanoparticles comprising second reactive moieties that comprise tetrazine groups, or by combining a reactive polymer comprising first reactive moieties that comprise tetrazine groups and gold nanoparticles comprising second reactive moieties that comprise strained alkyne groups. The reactive polymer and the gold nanoparticles are combined under conditions such that the strained alkyne and tetrazine groups crosslink with one another. Such crosslinked reaction products can be formed in vivo or ex vivo.

In some embodiments, the present disclosure provides a crosslinked reaction product of a reactive polymer comprising reactive moieties that comprise thiol groups and gold nanoparticles that are stabilized with one or more stabilization agents that exhibit a lower affinity for gold than the thiol groups. In some embodiments, the present disclosure provides a system for forming a crosslinked reaction product by combining a reactive polymer comprising reactive moieties that comprise thiol groups with gold nanoparticles that are stabilized with one or more stabilization agents that exhibit a lower affinity for gold than the thiol groups. The reactive polymer and the gold nanoparticles are combined under conditions such that the thiol groups react with the gold nanoparticles to form gold-sulfur bonds. In certain embodiments, those conditions comprise an environment having a basic pH, for example, a pH ranging from 8.0 to 11.0. Such crosslinked reaction products can be formed in vivo or ex vivo.

In various embodiments, the crosslinked reaction products of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, a system is provided that comprises (a) a first composition that comprises a reactive polymer as described hereinabove and (b) a second composition that comprises gold nanoparticles as described herein, wherein the system is configured to deliver the reactive polymer and the gold nanoparticles under conditions such that covalent crosslinks are formed between the reactive polymer and the gold nanoparticles.

The first composition may be a first fluid composition comprising the gold nanoparticles or a first dry composition that comprises the gold nanoparticles, to which a suitable fluid such as water for injection, saline, etc. can be added to form a first fluid composition. In addition to the gold nanoparticles, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the gold nanoparticles with a second fluid comprising the reactive polymer. Upon mixing the first and second fluid compositions, the gold nanoparticles crosslink with the reactive polymer, forming a crosslinked product. The first and second fluid compositions may be combined to form radiopaque crosslinked hydrogels, either in vivo or ex vivo.

In some embodiments, the gold nanoparticles are initially combined with the reactive polymer under conditions where crosslinking between the reactive polymer and the gold nanoparticles is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the gold nanoparticles and the reactive polymer, thereby forming a crosslinked product.

In some embodiments, the system comprises (a) a first composition that comprises gold nanoparticles as described hereinabove, (b) a second composition that comprises a reactive polymer as described hereinabove, and (c) a third composition, specifically, an accelerant composition, that contains an accelerant that is configured to accelerate a crosslinking reaction between the gold nanoparticles and the reactive polymer.

The first composition may be a first fluid composition comprising the gold nanoparticles that is buffered to an acidic pH or a first dry composition that comprises the gold nanoparticles, to which a suitable fluid such as water for injection, saline, an acidic buffer solution, etc. can be added to form a first fluid composition comprising the gold nanoparticles that is buffered to an acidic pH. In some embodiments, for example, the acidic buffering composition may comprise monobasic sodium phosphate, among other possibilities. The first fluid composition comprising the gold nanoparticles may have a pH ranging, for example, from about 3 to about 5. In addition to the gold nanoparticles, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the gold nanoparticles that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the gold nanoparticles that is buffered to an acidic pH and the second composition comprises a dry composition that comprises the reactive polymer. The first composition may then be mixed with the second composition to provide a prepared fluid composition that is buffered to an acidic pH and comprises the gold nanoparticles and the reactive polymer. In a particular example, a syringe may be provided that contains the first fluid composition comprising the gold nanoparticles that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the gold nanoparticles and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the gold nanoparticles and the reactive polymer as described above, and a fluid accelerant composition that is buffered to basic pH as described above, may be combined form radiopaque crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque gold particles that are present), for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises gold nanoparticles as described above and a second reservoir that contains a second fluid composition that comprises a reactive polymer as described above, wherein the first and second fluid compositions form a crosslinked product upon mixing. In some embodiments, the system may include a delivery device that comprises a first reservoir that contains a first fluid composition that comprises the gold nanoparticles and the reactive polymer and is buffered to an acidic pH, such as the prepared fluid composition previously described, and a second reservoir that contains second fluid composition, such as the fluid accelerant composition previously described.

In either case, during operation, the first fluid composition and second fluid composition are dispensed from the first and second reservoirs and combined, whereupon the gold nanoparticles and the reactive polymer and crosslink with one another to form a radiopaque crosslinked hydrogel.

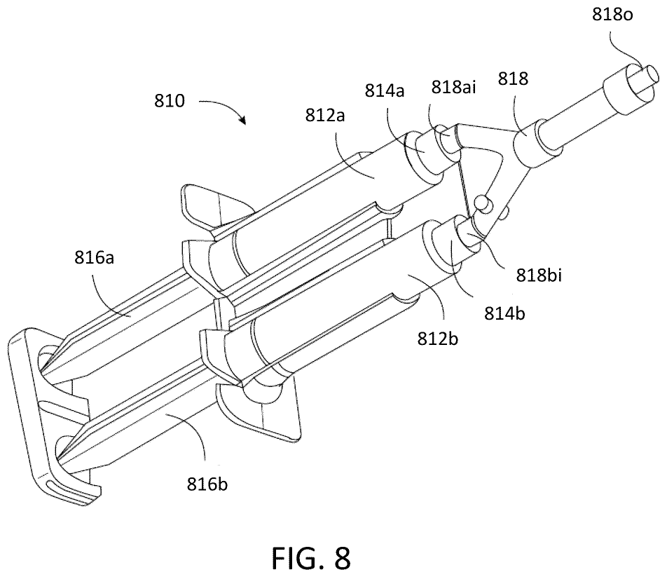

In particular embodiments, and with reference to FIG. 8, the system may include a delivery device 810 that comprises a double-barrel syringe, which includes first barrel 812a having a first barrel outlet 814a, which first barrel contains the first composition, a first plunger 816a that is movable in the first barrel 812a, a second barrel 812b having a second barrel outlet 814b, which second barrel 812b contains the second composition, and a second plunger 816b that is movable in the second barrel 812b. In some embodiments, the device 810 may further comprise a mixing section 818 having a first mixing section inlet 818ai in fluid communication with the first barrel outlet 814a, a second mixing section inlet 818bi in fluid communication with the second barrel outlet, and a mixing section outlet 8180.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a radiopaque crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

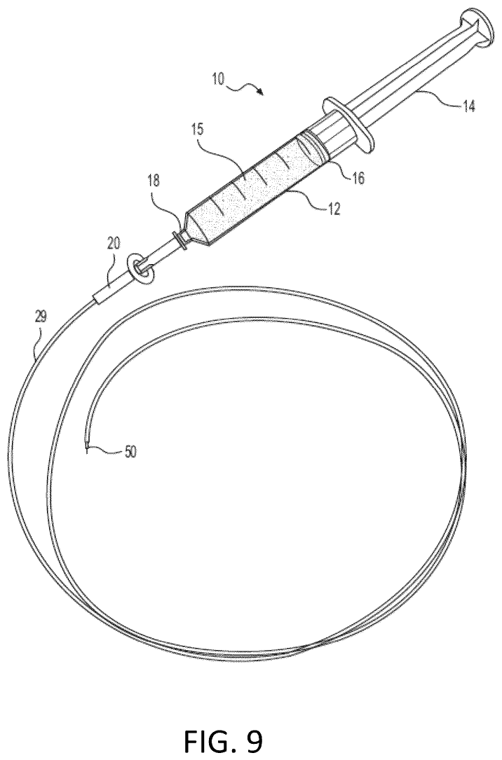

FIG. 9 illustrates a syringe 10 providing a reservoir for a radiopaque crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque crosslinked hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

After administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system for forming a hydrogel composition that comprises (a) a reactive polymer comprising a plurality of hydrophilic polymer segments and a plurality of first reactive moieties and (b) gold nanoparticles, wherein the system is configured to deliver the reactive polymer and the gold nanoparticles under conditions such that covalent crosslinks are formed between the reactive polymer and the gold nanoparticles.

2. The system of claim 1, wherein the reactive polymer is a multi-arm polymer that comprises three or more polymer arms linked to a core region, each arm comprising one of the plurality of hydrophilic polymer segments and one of the plurality of first reactive moieties.

3. The system of claim 1, wherein the reactive polymer is a multi-arm polymer that comprises three or more polymer arms linked to a core region, each arm comprising a cyclic anhydride residue disposed between one of the plurality of hydrophilic polymer segments and one of the plurality of first reactive moieties.

4. The system of claim 2, wherein the core region comprises a polyol residue.

5. The system of claim 1, wherein the hydrophilic polymer segments are selected from polyalkylene oxide segments, polyester segments, polyoxazoline segments, polydioxanone segments, and polypeptide segments.

6. The system of claim 1, wherein each of the hydrophilic polymer segments contains between 10 and 1000 monomer residues.

7. The system of claim 1, wherein the gold nanoparticles range from 1 nm to 2 micrometers in longest dimension.

8. The system of claim 1, wherein the first reactive moieties comprise thiol groups and the gold nanoparticles comprise a stabilization agent that exhibits a lower affinity for gold than the thiol groups.

9. The system of claim 8, wherein the stabilization agent is a surfactant.

10. The system of claim 1, wherein the gold nanoparticles comprise a plurality of second reactive moieties that form covalent crosslinks with the first reactive moieties.

11. The system of claim 10, wherein the first reactive moieties comprise a cyclic imide ester group and the second reactive moieties comprise a primary amine, thiol or hydroxyl group, or wherein the first reactive moieties comprise a primary amine, thiol or hydroxyl group and the second reactive moieties comprise a cyclic imide ester group.

12. The system of claim 10, wherein the first reactive moieties comprise a strained alkyne group and the second reactive moieties comprise an azide group, or wherein the first reactive moieties comprise an azide group and the second reactive moieties comprise a strained alkyne group.

13. The system of claim 10, wherein the first reactive moieties comprise a strained alkene group and the second reactive moieties comprise tetrazine a group, or wherein the first reactive moieties comprise a tetrazine group and the second reactive moieties comprise a strained alkene group.

14. The system of claim 1, further comprising a delivery device.

15. A method of treatment comprising administering to a subject a mixture that comprises (a) a reactive polymer comprising a plurality of hydrophilic polymer segments and a plurality of first reactive moieties and (b) gold nanoparticles, under conditions such that the reactive polymer and the gold nanoparticles crosslink after administration.

16. The method of claim 15, wherein the method comprises administering to the subject a first fluid composition that comprises the reactive polymer and a second fluid composition that comprises the gold nanoparticles.

17. The method of claim 15, wherein the method comprises administering to the subject a first fluid composition that comprises the reactive polymer and the gold nanoparticles and a second fluid composition that comprises an accelerant that accelerates formation of the covalent crosslinks.

18. The method of claim 15, wherein the first fluid composition and the second fluid composition are delivered using a double barrel syringe.

19. A radiopaque crosslinked hydrogel composition comprising a crosslinked reaction product of (a) a reactive polymer comprising a plurality of hydrophilic polymer segments and a plurality of first reactive moieties and (b) gold nanoparticles.

20. A method of treatment comprising administering to a subject the radiopaque crosslinked hydrogel composition of claim 19.
